(19) United States
(12) Patent Application Publication
(10) Pub. No.: US 2026/0327657 A1
(43) Pub. Date: Oct. 22, 2026

(54) Compositions And Methods For Nongenetic Cell Modification

(71) Applicant: The University Of North Carolina At Greensboro, Greensboro, NC (US)

(72) Inventors: Daniel J. C. Herr, Greensboro, NC (US); Tetyana Ignatova, Greensboro, NC (US); Adeyinka O. Adesina, Greensboro, NC (US)

(21) Appl. No.: 19/106,843

(22) PCT Filed: Aug. 31, 2023
(86) PCT No.: PCT/US23/31766

§ 371 Date: Feb. 26, 2025

Related U.S. Application Data
(60) Provisional application No. 63/403,434, filed on Sep. 2, 2022.

(51) Int. Cl. C12N 15/87

(52) U.S. Cl. CPC C12N 15/87

(57) ABSTRACT

Described are compositions and methods for delivering peptides to cells using single wall carbon nanotubes non-covalently associated with a single-stranded nucleic acid. For example, disclosed are compositions having a single wall carbon nanotube (SWCNT), a single-stranded nucleic acid, and at least one peptide as well as methods of making and using such compositions. In some instances, the single-stranded nucleic acid may be non-covalently attached to an outer surface of the nanotube. Also in some instances, the peptide may bind to the 5′ and/or 3′ terminus of the single-stranded nucleic acid. The compositions and methods may provide therapeutic benefits. For example, such compositions and methods may be used in the field of biomedicine as for example, oncology.

### FIELD OF THE INVENTION

The invention relates to compositions and methods for nongenetic cell modification.

### BACKGROUND

Single wall carbon nanotubes (SWCNT) are 0.4 to 2 nm diameter tubular structures that can be formed by individual graphene cylinders. Interest in using SWCNTs in biomedical or nanomedicine applications has grown largely due to their low toxicity in various living cells and lack of immunogenicity. The mechanical, optical, and electronic properties of SWCNTs also make them useful in improving imaging systems and devices to carry drugs to a targeted location. Hirlekar et al., (2009) Carbon nanotubes and its applications: a review, Asian J. of Pharma. And Clin Res. 2:17-27; Schmidt et al. (2015) Nanomaterials—Tools, Technology and Methodology of Nanotechnology Based Biomedical Systems for Diagnostics and Therapy, Biomedicines 3(3)-203-223. Additionally, the ultra-small size, large surface area to mass ratio, and high reactivity of SWCNTs allow them to adsorb or conjugate with a wide variety of therapeutic molecules such as drugs, proteins, antibodies, nucleic acids, and enzymes. Kumar et al. Carbon-Based Nanomaterials, (2016) Essentials in Nanoscience, John Wiley & Sons, Inc.: Hoboken, NJ, U.S.A., 189-236.

SWCNTs can be functionalized by covalent and non-covalent interactions to impart desirable properties. For example, SWCNTs are insoluble in aqueous solutions due to their highly hydrophobic surfaces, but they can be functionalized to be more soluble by using covalent interactions such as oxidation with a strong acid to produce surface carboxyl groups, or through noncovalent interactions such as DNA or RNA wrapping around the surface of the nanotube.

There is growing interest in developing SWCNTs as useful and effective vehicles for the delivery of molecules into and nongenetic modification of cells. The present disclosure describes such novel uses for SWCNTs as vehicles for the delivery of peptides into cells.

### BRIEF SUMMARY

Disclosed here in are compositions and methods for nongenetic cell modification. The disclosure may be embodied in a variety of ways. For example, in one embodiment, provided herein is a composition comprising a single wall carbon nanotube (SWCNT), a single-stranded nucleic acid, and at least one peptide, wherein the single-stranded nucleic acid is non-covalently bound to an outer surface of the nanotube, and wherein the peptide binds to the 5′ and/or 3′ terminus of the single-stranded nucleic acid.

In some embodiments, the single-stranded nucleic acid wraps around at least part of an outer surface of the SWCNT.

In some embodiments, the single-stranded nucleic acid non-covalently attached to a SWCNT is bound to more than one peptide via the 5′ and/or 3′ terminus of the single-stranded nucleic acid. In some embodiments, the single-stranded nucleic acid is electrostatically attached to the SWCNT. In some embodiments, the peptide binds to the 5′ or 3′ terminus of the single-stranded nucleic acid without touching the SWCNT.

In some embodiments, the nucleic acid is DNA. In some embodiments, the nucleic acid is RNA. In some embodiments, the nucleic acid is covalently or non-covalently functionalized. In some embodiments, the nucleic acid is DNA with a sequence of alternating guanine and thymine residues (i.e., G's and T's). The size of the nucleic acid used, may, in certain embodiments, depend on the size of the SWCNT. In some embodiments, the nucleic acid is 15 nucleotides long. Or, a nucleic acid of other length may be used. Additionally, and/or alternatively, the size of the SWCNT may be varied depending upon the desired use. In some embodiments, the SWCNT has a diameter of 0.4 to 2 nm. In some embodiments, the SWCNT has a length of 100 nm to 5 microns. Or, other lengths may be used.

In some instances, the cell is a prokaryotic cell. In some embodiments, the cell is a eukaryotic cell. In some embodiments, the cell is an algal cell. In some embodiments, the cell is a mammalian cell. In some instances, the cell is an isolated human cell. In some embodiments, the cell is a human cell within a human body.

As disclosed herein, the size of various components of the disclosed composition may vary depending on the application. In some instances, the single-stranded nucleic acid non-covalently attached to a SWCNT without an attached peptide is less than 60 nm in diameter along at least one axis. Or, other sizes may be used. In some embodiments, the peptide has a diameter less than 60 nm. Or, other sizes may be used. In some embodiments, the peptide is nblA. In some embodiments, the single-stranded nucleic acid is able to detach from the SWCNT at a generally neutral pH. For example, in some embodiments, the single-stranded nucleic acid is able to detach from the SWCNT at a pH of 5.5 to 7.7. In some embodiments, the single-stranded nucleic acid is able to detach from the SWCNT in response to a solvatochromatic shift or Manning Ossawa condensation parameter. In some embodiments, the single-stranded nucleic acid has a nanobrush or nanotree structure.

Also provided are methods for delivering a peptide into a cell comprising contacting a single-stranded nucleic acid non-covalently attached to a single wall carbon nanotube (SWCNT) bound to at least one peptide via the 5′ and/or 3′ terminus of the single-stranded nucleic acid with a cell.

In some instances, the single-stranded nucleic acid wraps around at least part of an outer surface of the SWCNT.

In some embodiments of the disclosed methods, the single-stranded nucleic acid non-covalently attached to a SWCNT is bound to more than one of the at least one peptide via the 5′ and/or 3′ terminus of the single-stranded nucleic acid. In some embodiments, the single-stranded nucleic acid is electrostatically attached to the SWCNT. In some embodiments, the peptide binds to the 5′ or 3′ terminus of the single-stranded nucleic acid without touching the SWCNT.

In some embodiments of the disclosed methods, the nucleic acid is DNA. In some embodiments, the nucleic acid is RNA. In some embodiments, the nucleic acid is covalently or non-covalently functionalized. In some embodiments, the nucleic acid is DNA with a sequence of alternating G's and T's. In some embodiments, the nucleic acid is 15 nucleotides long. In some embodiments, the SWCNT has a diameter of 0.4 to 2 nm. In some embodiments, the SWCNT has a length of 100 nm to 5 microns.

In some instances, the cell is a prokaryotic cell. In some embodiments, the cell is a eukaryotic cell. In some embodiments, the cell is an algal cell. In some embodiments, the cell is a mammalian cell. In some embodiments, the cell is an isolated human cell. In some embodiments, the cell is a human cell within a human body.

In some instances, the single-stranded nucleic acid non-covalently attached to a SWCNT without an attached peptide is less than 60 nm in diameter along at least one axis. In some embodiments, the peptide has a diameter less than 60 nm. In some embodiments, the peptide is nblA. In some embodiments, the single-stranded nucleic acid is able to detach from the SWCNT at pH of 5.5 to 7.7. In some embodiments, the single-stranded nucleic acid is able to detach from the SWCNT in response to a solvatochromatic shift or Manning Ossawa condensation parameter. In some embodiments, the single-stranded nucleic acid has a nanobrush or nanotree structure.

Also provided are methods for treating a disease comprising contacting a cell with a peptide bound to a single-stranded nucleic acid that is non-covalently attached to an outer surface of a SWCNT. In certain embodiments, the peptide is a therapeutic peptide. In certain embodiments, the disease is cancer. In certain embodiments, the peptide is a chemotherapeutic peptide.

In certain embodiments, the SWCNT is a SWCNT as disclosed herein. For example, in some embodiments, the single-stranded nucleic acid wraps around at least part of an outer surface of the SWCNT. In certain embodiments, the SWCNT comprises a single-stranded nucleic acid, and at least one peptide, wherein the single-stranded nucleic acid is non-covalently bound to the outer surface of the nanotube, and wherein the therapeutic peptide binds to the 5′ and/or 3′ terminus of the single-stranded nucleic acid.

In some embodiments, the single-stranded nucleic acid non-covalently attached to a SWCNT is bound to more than one therapeutic peptide via the 5′ and/or 3′ terminus of the single-stranded nucleic acid. In some embodiments, the single-stranded nucleic acid is electrostatically attached to the SWCNT. In some embodiments, the therapeutic peptide binds to the 5′ or 3′ terminus of the single-stranded nucleic acid without touching the SWCNT.

In some instances, the nucleic acid is DNA. In some embodiments, the nucleic acid is RNA. In some embodiments, the nucleic acid is covalently or non-covalently functionalized. In some embodiments, the nucleic acid is DNA with a sequence of alternating G's and T's. In some embodiments, the nucleic acid is 15 nucleotides long. In some embodiments, the SWCNT has a diameter of 0.4 to 2 nm. In some embodiments, the SWCNT has a length of 100 nm to 5 microns. Or, as disclosed herein, depending on the application (e.g., cell type being treated) various sizes for the SWCNT and nucleic acid may be used.

In some embodiments, the cell is a eukaryotic cell. In some embodiments, the cell is an isolated human cell. In some embodiments, the cell is a human cell within a human body.

In some instances, the single-stranded nucleic acid non-covalently attached to a SWCNT without an attached therapeutic peptide is less than 60 nm in diameter along at least one axis. In some embodiments, the therapeutic peptide has a diameter less than 60 nm. Or, as disclosed herein, depending on the application (e.g., cell type being treated) various sizes for the SWCNT and peptide may be used. In some embodiments, the single-stranded nucleic acid is able to detach from the SWCNT at pH of about 5.5 to 7.7 (e.g., generally neutral pH). In some embodiments, the single-stranded nucleic acid is able to detach from the SWCNT in response to a solvatochromatic shift or Manning Ossawa condensation parameter. In some embodiments, the single-stranded nucleic acid has a nanobrush or nanotree structure.

### BRIEF DESCRIPTION OF THE DRAWINGS

FIGS. 1A-1B show cryo-EM images of Synechococcus elongatus bacteria. FIG. 1A shows S. elongatus bacteria exposed to a solution containing a SWCNT attached to a nucleic acid 15 nucleotides long, comprising alternating G's and T's, wherein the nucleic acid is attached to an nblA peptide (nblA-GT15-SWCNT) in accordance with an embodiment of the disclosure. FIG. 1B shows resulting structures that appear to be carbon nanotubes (arrows) penetrating the outer membrane of S. elongatus in accordance with an embodiment of the disclosure. Circles highlight what appear to be insertion points.

FIG. 2 shows the impact of DNA wrapped CNTs on the internal processes of bacteria, e.g., S. elongatus. The left panel shows fluorescence intensity measurements of cyanobacteria (SE) at the resonance excitation of phycolibisome (546 nm) after 60 hours of starvation in accordance with an embodiment of the disclosure. The first column is a control cell. The second column is a cell incubated with nblA-DNA-SWCNT. The third and fourth columns are negative control cells with either no nblA peptide or no DNA/SWCNT. SE=Synechococcus elongatus; DNA/SWCNT=DNA-SWCNT; nblA+DNA/SWCNT=nblA hybrid=nblA-DNA-SWCNT. The right panel shows the use of fluorescence intensity to compare the kinetics of phycobilisome degradation over time for a control cell and a cell incubated with nblA-DNA-SWCNT hybrid in accordance with an embodiment of the disclosure. Relative photon counts are indicated as arbitrary (“arb”) units.

FIG. 3 left panel shows the ratio of phycobilisome fluorescence degradation rates in accordance with an embodiment of the disclosure. The right panel shows the corresponding rates of ATP production shown in accordance with an embodiment of the disclosure. SE is S. elongatus cells; SE+hybrid is S. elongatus cells incubated with nblA-DNA-SWCNT.

### DETAILED DESCRIPTION

### Terms and Concepts

The terms and concepts discussed below are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Known methods and techniques are generally performed according to conventional methods well-known and as described in various general and more specific references, unless otherwise indicated. The nomenclatures used in connection with the laboratory procedures and techniques described in the present disclosure are those well-known and commonly used.

As used in the present disclosure, the terms “a”, “an”, and “the” can refer to one or more unless specifically noted otherwise. Unless otherwise dictated by context, singular terms shall include pluralities, and plural terms shall include the singular.

The use of the term “or” is used to mean “and/or,” unless explicitly indicated to refer to alternatives only, or the alternatives are mutually exclusive, although the disclosure supports a definition that refers to only alternatives and “and/or.” As used in the present disclosure “another” can mean at least a second or more.

As used in the present disclosure, and unless otherwise indicated, the terms “include,” “including,” and, in some instances, similar terms (such as “have” or “having”) mean “comprising.”

When a numerical range is provided in the present disclosure, the numerical range includes the range endpoints unless otherwise indicated. Unless otherwise indicated, numerical ranges in the present disclosure include all values and subranges, as if explicitly written out.

The terms “about” as used in the present disclosure, shall generally mean an acceptable degree of error for the quantity measured, given the nature or precision of the measurements. Exemplary degrees of error are within 20%, 10%, 9%, 8%, 7%, 6%, 5%, 4%, 3%, 2%, or 1% of a given value or range of values. For example, any reference to “about X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. In another example, the term “about” in relation to a reference numerical value can include a range of values plus or minus 10%, 9%, 8%, 7%, 6%, 5%, 4%, 3%, 2%, or 1% from that value. Thus, the expression “about X” is intended to describe a claim limitation of, for example, “0.98X.” Numerical quantities given in the present disclosure are approximate unless stated otherwise, meaning that the term “about” can be inferred when not expressly stated. When the term “about” is applied to the beginning of a numerical range, it applies to both ends of the range. Where a series of values is prefaced with the terms “about,” this term is intended to modify each value included in the series.

The term “nucleic acid” as used herein refers to a polymer containing at least two deoxyribonucleotides or ribonucleotides includes DNA and RNA. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid.

“Nucleotides” contain a sugar deoxyribose (DNA) or ribose (RNA), a base, and a phosphate group. Nucleotides are linked together through the phosphate groups. “Nucleobases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The terms “peptide,” “polypeptide” or “protein” are used to refer polymer of amino acids linked by native amide bonds and/or non-native amide bonds. Peptides, polypeptides or proteins may include moieties other than amino acids (for example, lipids or sugars). Peptides, polypeptides or proteins may be produced synthetically or by recombinant technology.

For a molecule to be covalently or non-covalently “functionalized,” it can be attached covalently or non-covalently to another molecule or molecules that impart a new function such as increased stability or enhanced cell internalization. Non-limiting examples of functionalized DNA can be found in, for example, Nicholson et al. (2020). DNA Nanostructures and DNA-Functionalized Nanoparticles for Cancer Theranostics. Advanced Science. 7:2001669.

“Nanobrush” or “nanotree” describes a tree-like or brush-like structure that may be adopted by nanoparticles that are visible by electron microscopy. See, for example, Sun et al. (2020). PNAS. Hierarchical supramolecular assembly of a single peptoid polymer into a planar nanobrush with two distinct molecular packing motifs. 117:31639-47.

A “therapeutic peptide” is a bioactive naturally occurring or synthetic peptide less than 100 amino acids long. Examples of therapeutic peptides include peptides that deliver treatments to cells or peptides that affect signal transduction pathways, some of which are described in Wang et al. (2022). Therapeutic peptides: current applications and future directions. Nature. Therapeutic peptides include chemotherapeutic peptides, sometimes referred to as anti-cancer peptides. https://doi.org/10.1038/s41392-022-00904-4. Non-limiting examples of chemotherapeutic peptides are described in Xie et al. (2020). Anti-cancer peptides: classification, mechanism of action, reconstruction and modification. Open Biology. https://doi.org/10.1098/rsob.200004.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, can include treatment resulting in inhibiting the disease, i.e., arresting its development; and relieving the disease, i.e., causing regression of the disease. For example, in the case of dilated cardiomyopathy, a response to treatment can include complete response, partial response, stable disease, progressive disease, progression free survival, or overall survival.

### Compositions and Methods

The present disclosure provides single-stranded nucleic acid wrapped single wall carbon nanotubes that can be used as cellular delivery vehicle. SCWNTs have been rarely used to deliver a peptide into a cell. SWCNTs wrapped with single-stranded nucleic acids have never been used previously in a complex with a peptide in order to facilitate delivery of the peptide. In some embodiments, the cellular delivery vehicle comprises a single wall carbon nanotube (SWCNT), a single-stranded nucleic acid, and at least one peptide, wherein the single-stranded nucleic acid is non-covalently attached to an outer surface of the nanotube, and wherein the peptide binds to the 5′ and/or 3′ terminus of the single-stranded nucleic acid. In certain embodiments, the single-stranded nucleic acid wraps around at least part of an outer surface of the SWCNT. In some embodiments, the single-stranded nucleic acid molecule non-covalently attached to a single wall carbon nanotube (SWCNT) is bound to more than one of the at least one peptide via the 5′ and/or 3′ terminus of the single-stranded nucleic acid molecule.

In some embodiments, the 5′ and 3′ termini of the single-stranded nucleic acid interact via electrostatic interactions and can create a binding site for a peptide. In some embodiments, the 5′ and 3′ termini of the single-stranded nucleic acid interact via electrostatic interactions and can create a binding site for more than one peptide. In some embodiments, the peptide selectively binds to 5′ or 3′ terminus on single-stranded nucleic acid without touching the SWCNT backbone.

In some embodiments, the single-stranded nucleic acid molecule is DNA. In some embodiments, the single-stranded nucleic acid molecule is RNA. In some embodiments, the nucleic acid is covalently or non-covalently functionalized. Both single-stranded DNA and RNA are capable of attaching to SWCNT. See, for example, Landry et al. (2015) Comparative Dynamics and Sequence Dependence of DNA and RNA Binding to Single Walled Carbon Nanotubes, J. Phys Chem Nanomaterials and Interfaces. 119:10048-58.

Compared to other polymers used, a nucleic acid may offer the advantage of defined length and sequence, high dispersion efficiency (i.e., up to 4 mg/mL), and well-developed chemistries for further functionalization of a nucleic acid-SWCNT hybrid through either covalent or non-covalent functionalization. See, e.g., Hu, et al. (2005), DNA Functionalized Single-Walled Carbon Nanotubes for Electrochemical Detection, The Journal of Physical Chemistry B, 109(43), 20072-20076.

Short single-stranded DNA includes a hydrophobic side which may attach itself to the hydrophobic end of a SWCNT. This is in contrast to double-stranded DNA which does not provide for π stacking for interacting with a SWCNT. For example, polyaromatic adsorption π-π stacking, CH-π stacking, protein adoption, and lipid adsorption, and van der Waals interactions may occur between single-stranded DNA and nanotubes. Mittal, V. (2011). Surface Modification of Nanotube Fillers. Wiley-VCH Verlag Gmbh & Co. KGaA; Bai, et al. (2017) Improving the filler dispersion and performance of silicone rubber/multi-walled carbon nanotube composites by noncovalent functionalization of polymethylphenylsiloxane. Journal of Materials Science 52(12), 7516-7529.

Each of the four nucleobases (guanine, cytosine, adenine, and thymine) may orient in distinct ways with respect to the single wall carbon nanotube's long axis. Hughes, et al. (2007). Optical absorption of DNA-carbon nanotube structures. Nano Lett. 7(5):1191-4. Also, various single-stranded DNA polymers of alternating sequences may facilitate the separation of nanotubes based on their electrostatic properties. Zheng, et al. (2003). Structure-Based Carbon Nanotube Sorting by Sequence-Dependent DNA Assembly. Science. Vol. 302, Issue 5650, pp. 1545-1548; Tu, et al. (2009). DNA Sequence Motifs for Structure-Specific Recognition and Separation of Carbon Nanotubes. Nature, 460(7252): 250-253. Thus, in certain embodiments, the nanotube's electronic state and the base composition of the nucleic acid may determine the properties of the resulting DNA-nanotube hybrid..

In some embodiments, the single-stranded DNA used in the methods described herein has a sequence of alternating guanine (G) and thymidine (T) bases. In certain embodiments, a nucleic acid with alternating sequences of G's and T's adheres favorably to SWCNTs. For example, either (GT)₁₅ DNA or (GU)₁₅ RNA wrapped around SWCNTs can act as an optical sensor for dopamine. [0047] In some embodiments, the nucleic acid is 10-30 nucleotides long, e.g., 10-15 nucleotides long, 15-20 nucleotides long, or 20-30 nucleotides long. Or, other sizes may be used depending upon the target and application. In some embodiments, the nucleic acid is 15 nucleotides long. In some embodiments, the nucleic acid has the sequence of 5′-GTGTGTGTGTGTGTG-3′ (SEQ ID NO: 1). In some embodiments, length of the nucleic acid allows both the binding and release of the wrapped single stranded nucleic acid molecule from around a SWCNT under physiologically relevant conditions.

In some embodiments, the diameter of a SWCNT is about 0.4-2 nm (depending on the chirality), e.g., 0.4-0.5 nm diameter, 0.5-0.6 nm diameter, 0.6-0.7 nm diameter, 0.7-0.8 nm diameter, 0.8-0.9 nm diameter, 0.9-1.0 nm diameter, 1.0-2.0 nm diameter. In some embodiments, the diameter of a SWCNT is <0.4 nm. In some embodiments, the diameter of a SWCNT is >2 nm. In some embodiments, the SWCNT has a length ranging from 100 nm to several microns long. In some embodiments, the SWCNT is 100 nm to 5 microns long, e.g., 100 nm long, 100-200 nm long, 200-300 nm long, 300-400 nm long, 400-500 nm long, 500-600 nm long, 600-700 nm long, 700-800 nm long, 800 nm to 1 micron long, 1-2 microns long, 2-3 microns long, 3-4 microns long, 4-5 microns long. Or, other sizes may be used depending upon the target and application.

The disclosed SWCNTs may be used with a variety of cell types. In some embodiments, the cell used in the methods described herein is a prokaryotic cell. In some embodiments, the cell is a eukaryotic cell. In some embodiments, the cell is an algal cell. In some embodiments, the cell is a mammalian cell. In some embodiments, the cell is an isolated human cell. In some embodiments, the cell is a human cell within a human body.

In certain embodiments, the single-stranded nucleic acid non-covalently attached to a SWCNT without an attached peptide is less than about 60 nm in diameter along at least one axis. The outer membranes of cells often contain about 60 nm to 150 nm diameter pores. In contrast, internal membranes of prokaryotic and eukaryotic cells, such as the nucleus, mitochondria, or thylakoid, are typically only permeable to small organic molecules. In certain embodiments, a SWCNT without an attached peptide passes through the outer cell membrane pore. In some embodiments, the peptide has a diameter smaller than the diameter of an outer cell membrane pore, permitting it to pass through the outer membrane pore with the SWCNT. In some embodiments, the SWCNT with an attached peptide is ≤60 nm in diameter along its shorter axis.

In some embodiments, a peptide attached to an SWCNT of the disclosure provides a biological activity. In some embodiments, the peptide is nblA. Or other peptides may be used. nblA is an approximately 60 amino acid intracellular cyanobacterial protein involved in degradation of phycobiliprotein subunits of the light-harvesting phycobilisome complex during periods of nitrogen starvation. Phicobilisomes are attached the cytoplasmic surface of the thylakoid membrane. nblA can interact with the alpha subunits of phycobiliproteins but can also interact with a chaperone of a cyanobacterial Clp protease. nblA-mediated degradation of phycobilisomes within cyanobacterial cells is associated with a change in fluorescence intensity of the cyanobacterial cells. Since ATP is the end-product of the photosynthetic activity, ATP production is reduced if the light-harvesting ability of the phycobilisomes is reduced. As described in Examples 3 and 4, cyanobacterial cells incubated with nblA-DNA-SWCNT exhibit a reduction in ATP production, as expected for loss of phycobilisome activity.

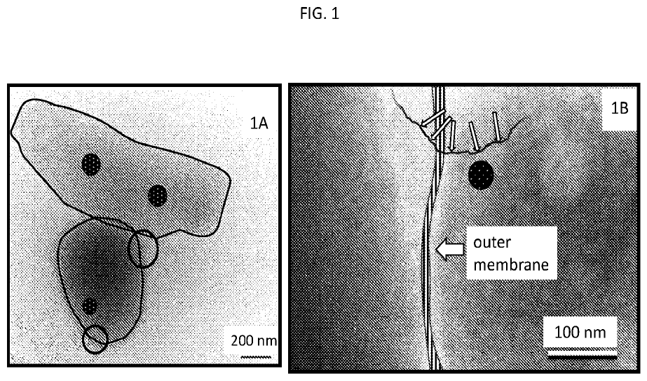

The effectiveness of a single-stranded nucleic acid wrapped SWCNT as a vehicle for gene or drug delivery hinges on the SWCNTs' ability to release DNA within the cell. In some embodiments, the single-stranded nucleic acid is able to detach from the SWCNT at a generally neutral pH. For example, in some embodiments, the single-stranded nucleic acid is able to detach from the SWCNT at a pH of 5.5 to 7.7. The pH inside a bacterial cell is approximately 7.5-7.7. Previous studies suggest that single-stranded DNA can be unwrapped from a SWCNT only at highly acidic and highly basic pHs. However, as shown in FIGS. 1-3, an nblA-DNA-SWCNT is able to enter the cell and release active nblA at approximately physiological pH. In some embodiments, single-stranded nucleic acid wrapped SWCNT comprises a single-stranded nucleic acid that is able to unwrap from the nanotube at a physiological pH between 5.5 and 7.7.

In certain embodiments, unwrapping of a SWCNT occurs in response to particular solvatochromic shifts and Manning Ossawa condensation parameters. For example, humidity and ammonia gas sensors have been developed using poly(methyl)methacrylate and poly (ethyleneoxide) films, making it possible to determine, for example, the concentration of water in organic solvents by using the strong solvatochromic sensitivity of betaine indicator dyes. Also, solvatochromic indicator dyes are sensitive to density fluctuations around solute molecules. See, e.g., Martins, L. R. (2003) Solvation dynamics of coumarin 153 in dimetheylsulfoxide-water mixtures: Molecular dynamics simulations, J. Chem. Phys. 118, 5955-5963. Also, for the sub-class of solution electrostatics problems that involve charged rods, Manning theory of counterion condensation (CC) may predict the distribution of small, mobile ions around polymers, colloids, biomolecules and biomembranes in solution. See, e.g., Manning, G. S. 1969a. Limiting laws and counterion condensation in polyelectrolyte solutions. I. Colligative properties. J. Chem. Phys. 51:924-933. In some embodiments, the single-stranded nucleic acid wrapped SWCNT comprises single-stranded DNA that is able to unwrap from the nanotube based on particular solvatochromic shifts and Manning Ossawa condensation parameters.

Single-stranded DNA sometimes becomes shorter during sonication and possesses less wrapping efficiency, leading to partial hybridization when within the proximity of another complementary single-stranded DNA. This can lead to the formation of ‘nanobrush and nanotrees.’ Such structures often have enhanced peptide binding. In some embodiments, ‘nanobrush or nanotree’ structures enhance binding of a peptide to the single-stranded nucleic acid-wrapped SWCNT.

Also provided are methods for delivering a peptide into a cell comprising contacting a single-stranded nucleic acid non-covalently attached to a single wall carbon nanotube (SWCNT) bound to a peptide via the 5′ and/or 3′ terminus of the single-stranded nucleic acid with a cell using any of the compositions disclosed herein.

In some embodiments, the single-stranded nucleic acid wraps around at least part of an outer surface of the SWCNT. In some embodiments, the single-stranded nucleic acid non-covalently attached to a SWCNT is bound to more than one peptide via the 5′ and/or 3′ terminus of the single-stranded nucleic acid. In some embodiments, the single-stranded nucleic acid is electrostatically attached to the SWCNT. In some embodiments, the peptide binds to the 5′ or 3′ terminus of the single-stranded nucleic acid without touching the SWCNT.

In some embodiments, the nucleic acid is DNA. In some embodiments, the nucleic acid is RNA. In some embodiments, the nucleic acid is covalently or non-covalently functionalized. In some embodiments, the nucleic acid is DNA with a sequence of alternating G's and T's. In some embodiments, the nucleic acid is 15 nucleotides long. In some embodiments, the SWCNT has a diameter of 0.4 to 2 nm. In some embodiments, the SWCNT has a length of 100 nm to 5 microns.

In some embodiments, the cell is a prokaryotic cell. In some embodiments, the cell is a eukaryotic cell. In some embodiments, the cell is an algal cell. In some embodiments, the cell is an isolated human cell. In some embodiments, the cell is a human cell within a human body.

In some embodiments, the single-stranded nucleic acid non-covalently attached to a SWCNT without an attached peptide is less than 60 nm in diameter along at least one axis. In some embodiments, the peptide has a diameter less than 60 nm. In some embodiments, the peptide is nblA.

In some embodiments, the single-stranded nucleic acid is able to detach from the SWCNT at pH 5.5 to 7.7. In some embodiments, the single-stranded nucleic acid is able to detach from the SWCNT in response to a solvatochromatic shift or Manning Ossawa condensation parameter. In some embodiments, the single-stranded nucleic acid has a nanobrush or nanotree structure.

In certain embodiments, the SWCNT may be used to deliver a therapeutic peptide to the cell or cells of a subject. For example, provided is a method for treating a disease, comprising contacting a cell with a therapeutic peptide bound to a single-stranded nucleic acid that is non-covalently attached to an outer surface of a SWCNT. In some embodiments of the disclosed methods, the single-stranded nucleic acid wraps around at least part of an outer surface of the SWCNT. In some instances, the single-stranded nucleic acid non-covalently attached to a SWCNT is bound to more than one of the at least one peptide via the 5′ and/or 3′ terminus of the single-stranded nucleic acid. In some instances, the single-stranded nucleic acid is electrostatically attached to the SWCNT. In some instances, the therapeutic peptide is bound to the 5′ or 3′ terminus of the single-stranded nucleic acid without touching the SWCNT. In some embodiments, the nucleic acid is DNA. In some embodiments, the nucleic acid is RNA. In various embodiments, the nucleic acid may be covalently or non-covalently functionalized. In some instances, the nucleic acid is DNA with a sequence of alternating G's and T's. In some embodiments, the nucleic acid is 15 nucleotides long. Or, other nucleic acid sizes may be used.

As disclosed herein, the size of the SWCNT used in the disclosed methods may be varied depending on the application, the size of the peptide and the target. In some embodiments, the SWCNT has a diameter of 0.4 to 2 nm. In some instances, the SWCNT has a length of 100 nm to 5 microns. Or, other sizes may be used.

In some embodiments, the cell is a eukaryotic cell. For example, the cell may be an isolated human cell. In some embodiments, the cell is a human cell within a human body.

In some embodiments, the single-stranded nucleic acid non-covalently attached to a SWCNT without an attached therapeutic peptide is less than 60 nm in diameter along at least one axis. In some instances, the therapeutic peptide has a diameter less than 60 nm. In some instances, the single-stranded nucleic acid is able to detach from the SWCNT at pH 5.5 to 7.7. In some embodiments, the single-stranded nucleic acid is able to detach from the SWCNT in response to a solvatochromatic shift or Manning Ossawa condensation parameter. In some instances, the single-stranded nucleic acid has a nanobrush or nanotree structure.

In certain embodiments, the human is a patient in need of therapy. For example, in certain embodiments, the peptide is a therapeutic peptide of the disclosed SWCNT. In certain embodiments, the disease is cancer, and the peptide is a chemotherapeutic peptide of the disclosed SWCNT.

### Examples

### Example 1. Materials and Methods

Formation of a Novel nblA-DNA-SWCNTs

Single-stranded DNA (Integrated DNA Technologies, Iowa IA, USA) and SWCNT (CoMoCat, Sigma Aldrich's Louis MO, USA) were mixed in a 19.75 mg to 0.1 mg weight ratio in a 1 mL volume of dissolved in cacodylate buffer. The mixture was sonicated using UPS200St with Vial Tweeter (Hielscher Ultrasound Technology, Teltow, Germany) on ice for 90 min at 40 W.

The sonicated sample was then centrifuged at 16300×g for 15 min (accuSpin micro17, Fisher Scientific, Lenexa, KS, USA), to remove large particulates, undispersed SWCNTs, and other residual impurities after which the supernatant was collected and used for subsequent experiments. The supernatant was sonicated again for 90 min before removing free single-stranded DNA that did not take part in the wrapping process. To remove free single-stranded DNA, the sample was centrifuged at 16300×g for 15 min with 100 kDa dialysis filter and resuspended in cacodylate buffer. To prepare the nblA-DNA-SWCNT hybrid, 2 μL of 0.1 mg/L nblA peptide (from MyBioSource, San Diego, CA) solution was carefully transferred into 0.5 mL of 0.03 mg/L DNA-SWCNT solution. The mixture was incubated for 30 minutes at 29 °C at rotation speed of 100 rpm.

### Cyanobacteria Cell Types and Cell Cultures

S. elongatus PC 9742 (from ATCC) was grown in BG- 11 medium at 29 °C with shaking at 100 rpm in a ratio of 99:1. Cultures were maintained in a Benchmark Incu-Shaker mini outfitted with 4 compact fluorescent lamp (CFL) natural-spectrum bulbs from Lithonia Lighting China, rated at 128 W. Growth assays used a total volume of 10 ml of culture in 25-ml conical flasks using a cork that had pores meant for CO₂ and O₂ exchange needed for the photosynthesis. All assays were conducted using nine replicates. Cell growth was monitored by measuring the optical density at 750 nm (OD₇₅₀).

### Cryo-EM Imaging

Cryo-EM imaging was used to localize SWCNT in bacteria. Bacteria were collected from media and deposited on TEM grid. Sample was at cryogenic temperature. Low-dose images were collected on ThermoFisher Talos Arctica 200 keV cryo-TEM equipped with a Gatan K3 camera.

### Fluorescence Intensity Measurements

Fluorescence was recorded in a 10-mm quartz cuvette in a Horiba Yvon Jobin Fluoro-Max 4 spectrofluorometer equipped with xenon lamp. Samples were stirred and kept at room temperature and ambient light before measurement. The short integration time (duration of flashes) allowed the temperature to stay relatively constant throughout the measurements. Measurements were taken in the 600-800 nm range, covering the resonance excitation of phycobilisomes at 548 nm. Intensity of fluorescence is in counts per second units but is reported as relative arbitrary units since there is no calibrated reference.

### Kinase Glo Assays

A hot water ATP extraction from the control S. elongatus and S. elongatus with nblA-DNA-SWCNT hybrids was performed, as described in Yang, N.-C., et al. (2002). A Convenient One-Step Extraction of Cellular ATP Using Boiling Water for the Luciferin-Luciferase Assay of ATP. Analytical Biochemistry, 306(2), 323-327. A kinase glo assay was used to trace depletion of ATP: the luminescent signal is correlated with the amount of ATP present and inversely correlated with the amount of kinase activity. See Veloria, J. R., et al. (2016). Optimization of a Luminescence-Based High-Throughput Screening Assay for Detecting Apyrase Activity. SLAS DISCOVERY: Advancing Life Sciences R&amp; D, 22(1), 94-101; see also Koresawa, M. et al. (2004). High-Throughput Screening with Quantitation of ATP Consumption: A Universal Non-Radioisotope, Homogeneous Assay for Protein Kinase. ASSAY and Drug Development Technologies, 2(2), 153-160. Serial dilutions of the ATP standard were prepared to plot standard calibration curve.

10 μL of each ATP standard with known concentrations and the extracted samples were transferred into the 96-well white plate, 10 μL Kinase-Glo™ reagent was added to each sample in triplicates. These samples were incubated at room temperature for 10 minutes and the Luminescence was read using Infinite M200 Pro Tecan Plate reader.

Example 2. Delivering nblA-DNA-SWCNT Hybrid Into the Thylakoid Membrane of the S. elongatus Bacteria and Suppressing the Photosynthetic Activities of the S. elongatus Bacteria.

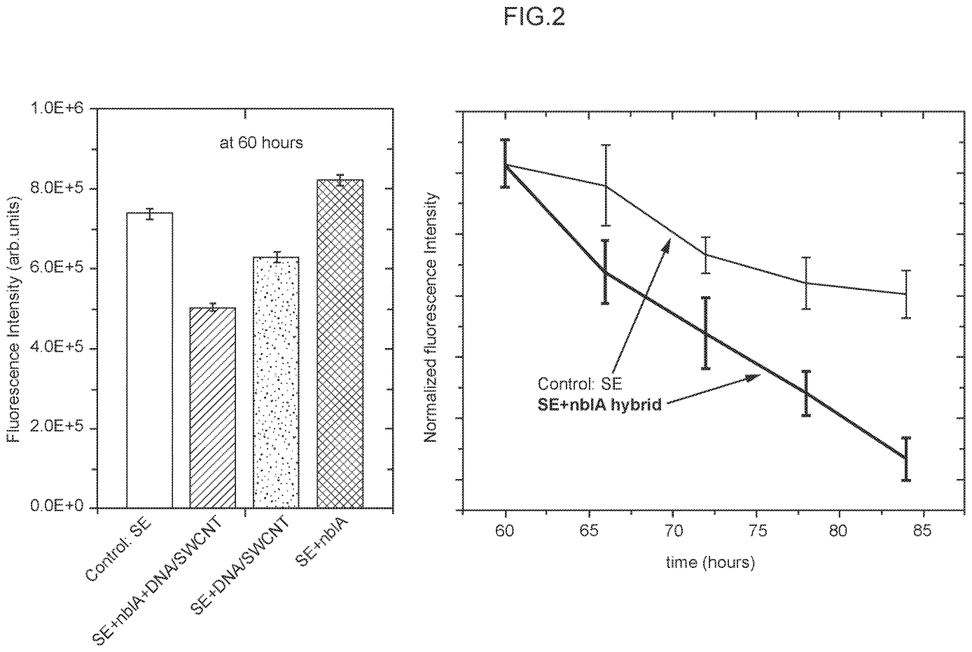

As noted above, SCWNTs have been rarely used to deliver a peptide into a cell. SWCNTs wrapped with single-stranded nucleic acids have never been used previously in a complex with a peptide in order to facilitate delivery of the peptide. Here, SWCNTs were used to deliver nblA, a non-bleaching peptide, into a cell with the help of a single-stranded DNA. FIG. 1 shows a cryo-EM image of Synechococcus elongatus bacteria incubated with nblA-DNA-SWCNT. The incubated cells have nanotube-like structures in their cell membranes, indicating that the carbon nanotubes attached to the single-stranded DNA and nblA are able to penetrate the cell membrane. As shown in FIG. 2, S. elongatus cells incubated with nblA-DNA-SWCNT also show indications of gradual phycobilisome degradation over time.

Example 3. Suppressing the Photosynthetic Activities of the S. elongatus Bacteria.

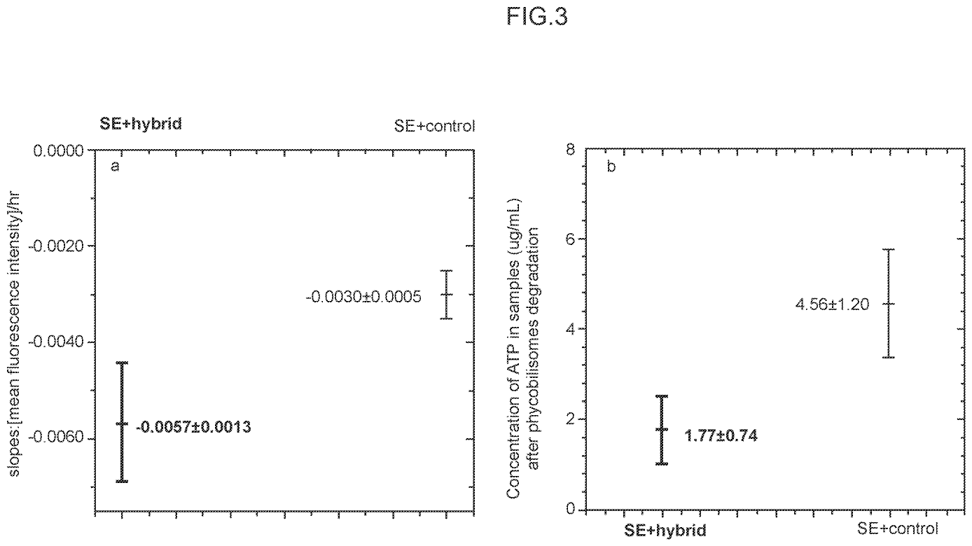

As shown in FIG. 3, using a Kinase-glo assay, the concentration of ATP in untreated S. elongatus cells was 4.56 (±1.20 , 2σ) μg/mL, but only 1.77 (±0.74 , 2σ) μg/mL in samples incubated with nblA-DNA-SWCNT, a 61% reduction. Specifically, a 2X fluorescence degradation rate correlated with 2X decrease in ATP production for treated bacteria versus untreated bacteria.

Although the foregoing inventions have been described in some detail by way of illustration and example for purposes of clarity of understanding, one of skill in the art will appreciate that certain changes and modifications can be practiced within the scope of the appended claims.

1. A composition comprising a single wall carbon nanotube (SWCNT), a single-stranded nucleic acid, and at least one peptide, wherein the single-stranded nucleic acid is non-covalently attached to an outer surface of the nanotube, and wherein the peptide binds to the 5′ and/or 3′ terminus of the single-stranded nucleic acid.

2. The composition of claim 1, wherein the single-stranded nucleic acid wraps around at least part of an outer surface of the SWCNT.

3. (canceled)

4. The composition of claim 1, wherein the single-stranded nucleic acid is electrostatically attached to the SWCNT.

5. (canceled)

6. The composition of claim 1, wherein the nucleic acid is DNA.

7. (canceled)

8. (canceled)

9. The composition of claim 6, wherein the nucleic acid is DNA comprising a sequence of alternating G's and T's.

10. The composition of claim 1, wherein the nucleic acid is 15 nucleotides long.

11. The composition of claim 1, wherein the SWCNT has a diameter of 0.4 nm to 2 nm.

12. The composition of claim 1, wherein the SWCNT has a length of 100 nm to 5 microns.

21. The composition claim 1, wherein the single-stranded nucleic acid is able to detach from the SWCNT at pH 5.5 to 7.7.

22. The composition of claim 1, wherein the single-stranded nucleic acid is able to detach from the SWCNT in response to a solvatochromatic shift or Manning Ossawa condensation parameter.

23. (canceled)

24. A method for delivering a peptide into a cell comprising contacting a single-stranded nucleic acid non-covalently attached to a single wall carbon nanotube (SWCNT) bound to a peptide via the 5′ and/or 3′ terminus of the single-stranded nucleic acid with a cell.

25. The method of claim 24, wherein the single-stranded nucleic acid wraps around at least part of an outer surface of the SWCNT.

26. (canceled)

27. The method of claim 24, wherein the single-stranded nucleic acid is electrostatically attached to the SWCNT.

28. (canceled)

29. The method of claim wherein the nucleic acid is DNA.

30. (canceled)

31. (canceled)

32. The method of claim 29, wherein the nucleic acid is DNA comprising a sequence of alternating G's and T's.

33. The method of claim 24, wherein the nucleic acid is 15 nucleotides long.

34. The method of claim 24, wherein the SWCNT has a diameter of 0.4 nm to 2 nm.

35. The method of claim 24, wherein the SWCNT has a length of 100 nm to 5 microns.

36.-43. (canceled)

44. The method of claim 24, wherein the single-stranded nucleic acid is able to detach from the SWCNT at pH 5.5 to 7.7.

45. The method of claim 24, wherein the single-stranded nucleic acid is able to detach from the SWCNT in response to a solvatochromatic shift or Manning Ossawa condensation parameter.

46. (canceled)

47. A method for treating a disease, comprising contacting a cell with a therapeutic peptide bound to a single-stranded nucleic acid that is non-covalently attached to an outer surface of a SWCNT.

48.-68. (canceled)
